(19) United States
(12) Patent Application Publication
(10) Pub. No.: US 2011/0124893 A1
(43) Pub. Date: May 26, 2011

(54) ANTIBIOTIC COMPOUNDS

(75) Inventors: Martin Decristoforo, Kundl (AT); Josef Wieser, Kundl (AT); Arthur Pichler, Kundl (AT); Andreas Hotter, Kundl (AT)

(73) Assignee: SANDOZ AG, Basel (CH)

(21) Appl. No.: 12/863,654

(22) PCT Filed: Jan. 19, 2009
(86) PCT No.: PCT/EP2009/050534

§ 371 Date: Sep. 27, 2010

(30) Foreign Application Priority Data
Jan. 23, 2008 (EP) 08150547.1

(51) Int. Cl. C07C 237/26

(52) U.S. Cl. 552/205

(57) ABSTRACT

The present invention relates to the new crystalline solid form XI of Tigecycline and a process of preparing the same. Form XI of Tigecycline is particularly suitable for the isolation of Tigecycline in the last step of the synthesis of Tigecycline. Further the present invention relates to a process of preparing amorphous Tigecycline by spray drying form XI or another crystalline form of Tigecycline.

### FIELD OF THE INVENTION

The present invention relates to the new crystalline solid form XI of Tigecycline and a process of preparing the same. Form XI of Tigecycline is particularly suitable for the isolation of Tigecycline in the last step of the synthesis of Tigecycline. Further the present invention relates to a process of preparing amorphous Tigecycline by spray drying form XI or another crystalline form of Tigecycline.

### BACKGROUND OF THE INVENTION

Tigecycline, (4S,4aS,5aR,12aS)-4,7-Bis(dimethylamino)-9-[[2-[(1,1-dimethylethyl)amino]acetyl]amino]-1,4,4a,5,5a,6,11,12a-octahydro-3,10,12,12a-tetrahydroxy-1,11-dioxo-2-naphthacene carboxamide (Fig. A), is a 9-t-butylglycylamido derivative of minocycline (Merck Index 14^{th} Edition, monograph number 9432, CAS Registry Number 220620-09-7). Compared to other tetracycline antibiotics Tigecycline is more active against tetracycline-resistant strains and also more tolerable. Tigecycline possesses activity against bacterial isolates containing the two major determinants responsible for tetracycline-resistance: ribosomal protection and active efflux of the drug out of the bacterial cell. Further Tigecycline has broad spectrum activity, as it is active against gram-positive pathogens (e.g. methicillin-resistant Staphylococcus aureus, vancomycin-resistant Enterococci), gram-negative pathogens (e.g. Acinetobacter baumannii, Stenotrophomonas maltophilia) and anaerobic pathogens. It is used for the treatment of complicated skin and skin structure infections and intra-abdominal infections [P. J. Petersen et al., Antimicrob. Agents and Chemoth. 43:738-744 (1999); R. Patel et al., Diagnostic Microbiology and Infectious Disease 38:177-179 (2000); H. W. Boucher et al., Antimicrob. Agents and Chemoth. 44:2225-2229 (2000); D. J. Biedenbach et al., Diagnostic Microbiology and Infectious Disease 40:173-177 (2001); P. J. Petersen et al., Antimicrob. Agents and Chemoth. 46:2595-2601 (2002); D. Milatovic et al., Antimicrob. Agents and Chemoth. 47:400-404 (2003); T. Hirata et al., Antimicrob. Agents and Chemoth. 48:2179-2184 (2004); G.A. Pankey Journal of Antimicrobial Chemotherapy 56:470-480 (2005); R. Harris et al., P&T 31:18-59 (2006)].

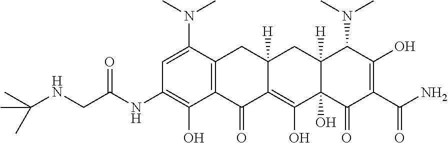

Figure A: Chemical Structure of Tigecycline

At the moment Tigecycline is only available as injectable antibiotic, as its oral bioavailability is very limited. The orange lyophilized powder or cake is available in 5 ml vials containing 50 mg of the amorphous agent [R. Harris et al., P&T 31:18-59 (2006)].

Patent application WO 2006/128150 discloses crystalline forms I, II, III, IV and V of Tigecycline and methods of their preparation. In WO 2007/127292 two crystalline forms but also an amorphous form of Tigecycline are disclosed as well as processes for their preparations. Nevertheless, there remains a need for alternative crystalline forms of Tigecycline, which have properties suitable for pharmaceutical processing on a commercial scale.

### SUMMARY OF THE INVENTION

The present invention relates to novel form XI of Tigecycline, a n-heptane solvate, characterized by an X-ray powder diffraction pattern with peaks at 3.9, 7.9, 8.8, 10.4, 10.9, 12.6, 13.5, 13.9, 15.8, 16.4, 17.8, 19.6, 20.3, 21.1 and 23.4 degrees 2-theta.

The present invention also provides a process of preparing form XI of Tigecycline comprising the steps of:
a) dissolving Tigecycline in methylene chloride
b) crystallizing Tigecycline form XI by the addition of n-heptane
c) stirring the solution at room temperature or below to effect complete crystallization
d) optionally isolating crystalline form XI of Tigecycline

In another embodiment, the present invention also provides a simple, cost-effective process of preparing amorphous Tigecycline by spray drying form XI or another crystalline form of Tigecycline. The experimental conditions of this process are easy to operate and suitable for large-scale production.

Other objects, features, advantages and aspects of the present invention will become apparent to those of skill from the following description. It should be understood, however, that the description and the following specific examples, while indicating preferred embodiments of the invention, are given by way of illustration only. Various changes and modifications within the spirit and scope of the disclosed invention will become readily apparent to those skilled in the art from reading the description and from reading the other parts of the present disclosure.

### BRIEF DESCRIPTION OF THE DRAWINGS

FIG. 1: X-ray powder diffraction pattern of form XI of Tigecycline

FIG. 2: Infrared spectrum of form XI of Tigecycline

FIG. 3: Differential scanning calorimetric curve of form XI of Tigecycline

FIG. 4: Thermogravimetric analysis curve of form XI of Tigecycline

FIG. 5: X-ray powder diffraction pattern of spray dried amorphous Tigecycline

FIG. 6: X-ray powder diffraction pattern of spray dried amorphous Tigecycline

### DETAILED DESCRIPTION OF THE INVENTION

As used herein the term “room temperature” indicates that the applied temperature is not critical and that no exact temperature value has to be kept. Usually, “room temperature” is understood to mean temperatures of about 15° C. to about 25° C. (see e.g. EU Pharmacopoeia 5.0, page 6).

The inventors of the present invention have identified a novel solvate of Tigecycline. The novel crystalline form is a n-heptane solvate and may be characterized e.g. by a typical X-ray powder diffraction pattern, infrared spectrum, a characteristic differential scanning calorimetric (DSC) curve or by a characteristic thermogravimetric analysis (TGA) curve. Each of these characteristics on its own is sufficient to unambiguously define and identify the new pseudo polymorph but they also may be combined with each other.

The present invention relates to a novel form XI of Tigecycline. Form XI of Tigecycline is a n-heptane solvate, hereinafter also referred to as “form XI” characterized by a X-ray powder diffraction pattern with peaks as shown in Table 1 at 3.9, 7.9, 8.8, 10.4, 10.9, 12.6, 13.5, 13.9, 15.8, 16.4, 17.8, 19.6, 20.3, 21.1 and 23.4 degrees 2-theta.

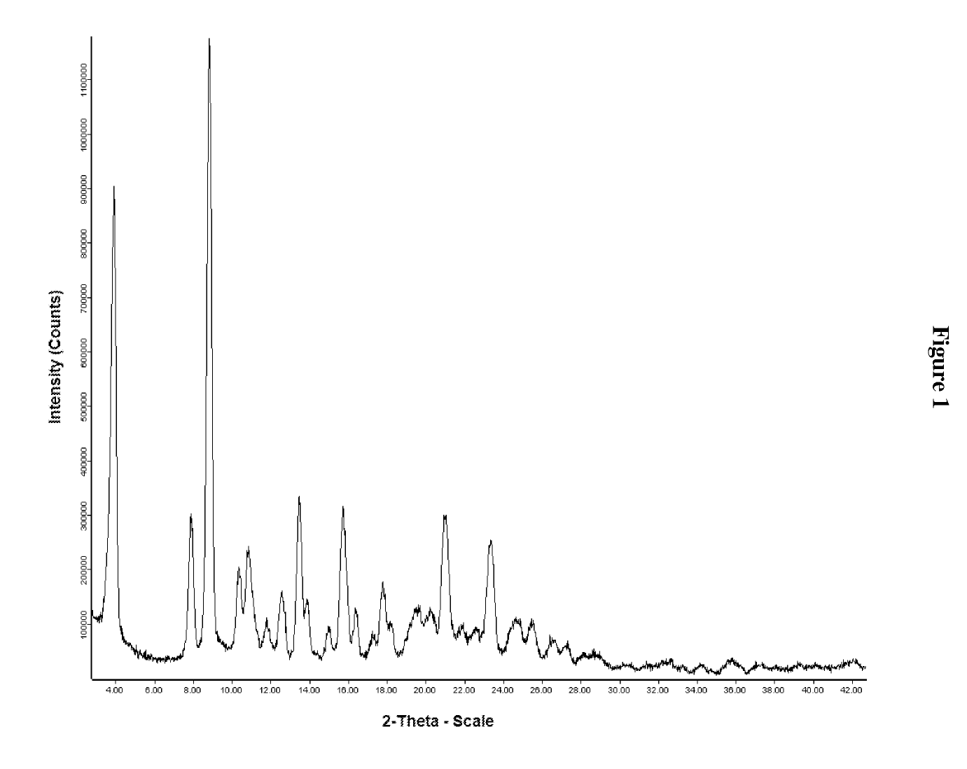

A characteristic X-ray powder diffraction pattern of form XI of Tigecycline is shown in FIG. 1 and some characteristic peaks are listed in table 1.

Accordingly, in a preferred embodiment, the present invention relates to a novel form XI of Tigecycline characterized by a X-ray powder diffraction pattern substantially in accordance with table 1 and FIG. 1.

The powder pattern of Form XI clearly can be distinguished from these of forms I, II, III, IV and V disclosed in patent application WO 2006/128150. For example, Form XI of Tigecycline possesses an intensive peak at 3.9 degrees 2-theta. This peak does not appear in any of the powder patterns of forms I to V from patent WO 2006/128150. Furthermore the whole powder pattern of form XI differs clearly from these of forms I, II, III, IV and V from WO 2006/128150.

In addition the powder pattern of form XI also can be distinguished from these of forms I and II from patent application WO 2007/127292. Form I of WO 2007/127292, for example, does not display peaks at 3.9, 7.9, 8.8, 10.4 and 10.8 degrees 2-theta, whereas form XI of the present invention does. On the other hand form I of WO 2007/127292 shows a characteristic peak at 11.4 degrees 2-theta, which is missing in the powder pattern of form XI.

Furthermore form XI of the present invention also shows a different powder pattern compared to the powder pattern of form II in WO 2007/127292, which does not display peaks at 3.9, 7.9, 8.8, 10.4 and 10.8 degrees 2-theta either. On the other hand form II of WO 2007/127292 shows characteristic peaks, for example, at 4.8, 6.4 and 6.8 degrees 2-theta, which are missing in the powder pattern of form XI.

Thus form XI of the present invention can be seen as a novel crystalline form of Tigecycline.

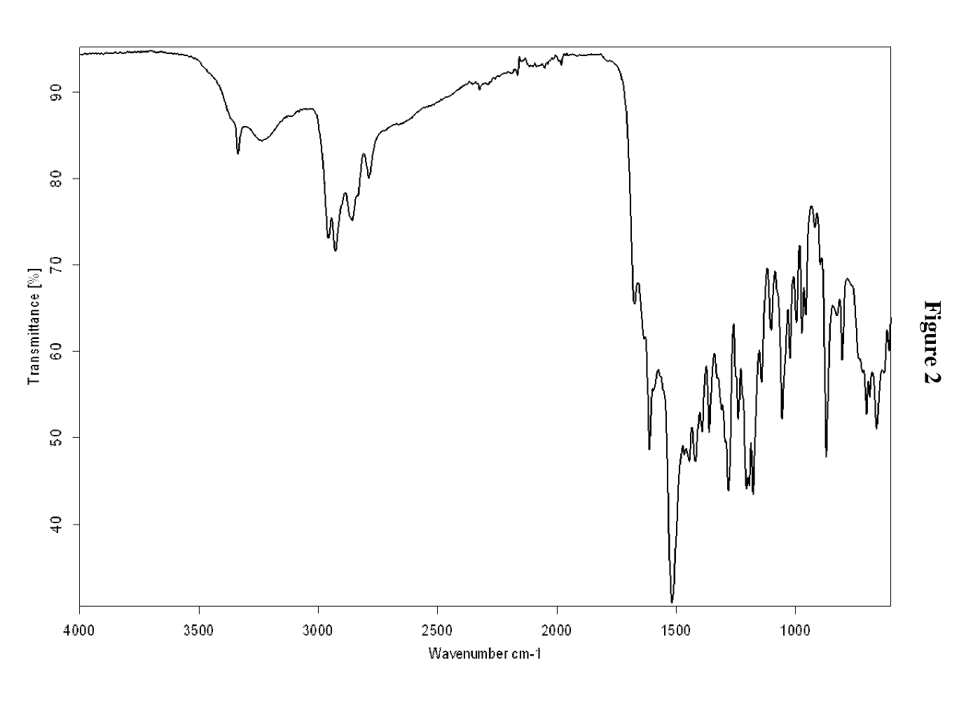

Form XI of Tigecycline may be also characterized by a typical infrared spectrum as shown in FIG. 2. Accordingly, in a further preferred embodiment, the present invention relates to form XI of Tigecycline characterized by an infrared spectrum substantially in accordance with FIG. 2. Characteristic bands are present at 2957, 2927, 2788, 1675, 1612, 1518, 1281, 1056 and 871 cm⁻¹.

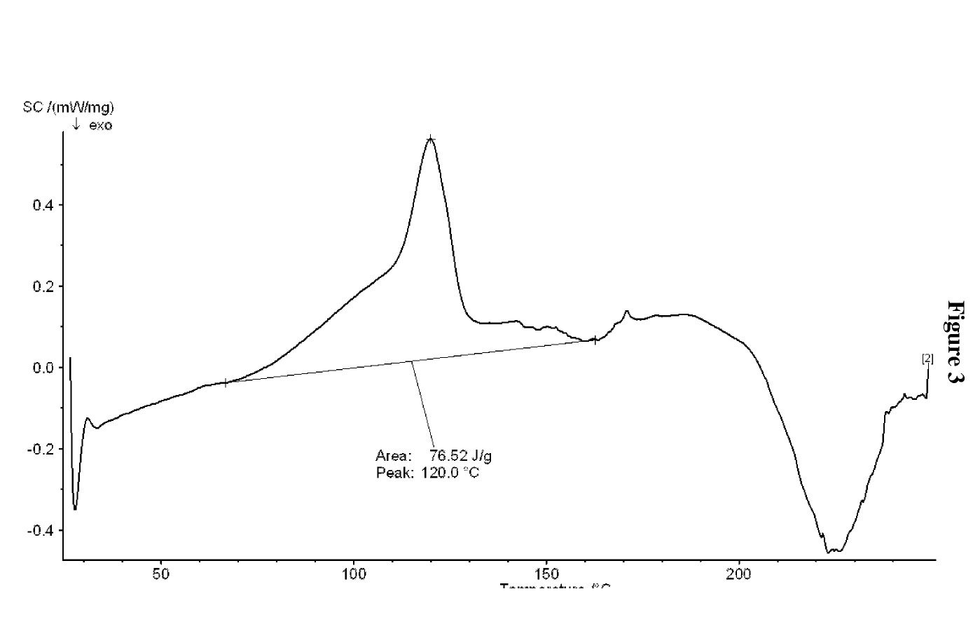

In addition, form XI of Tigecycline shows a characteristic DSC curve at a heating rate of 10° C./min. It can be seen in FIG. 3 that the DSC curve of form XI displays a broad endothermic peak, which is due to desolvation.

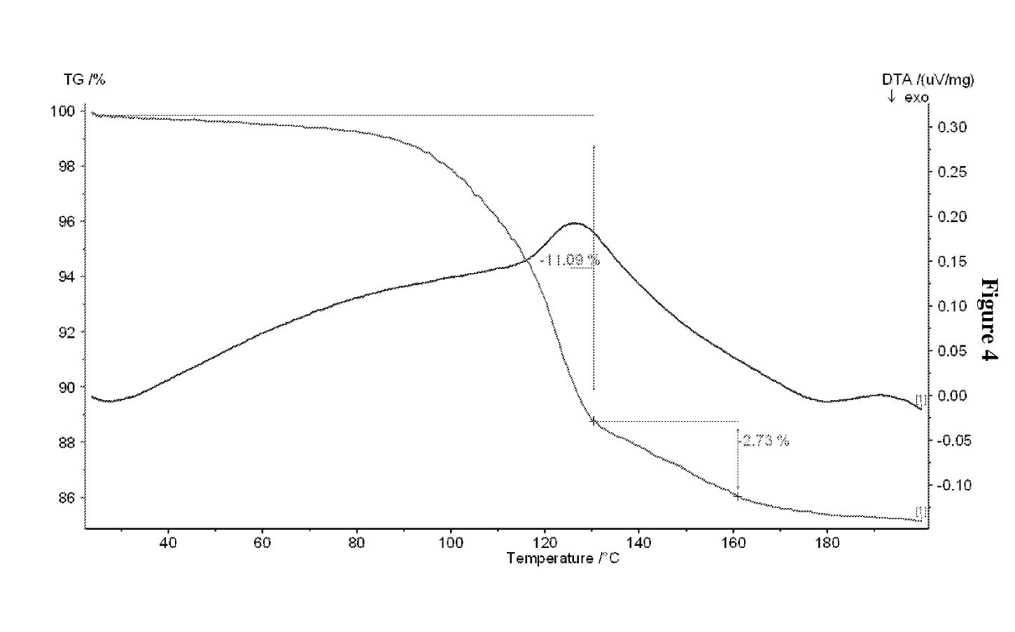

The TGA curve shown in FIG. 4 shows a total weight loss of about 13.8%, which is due to the desolvation process and shows good correspondence with GC-analysis.

TABLE 1
X-Ray Powder Diffraction (XRPD)
pattern of form XI of Tigecycline

| Angle | relative |
|---|---|
| [°2-theta] | Intensity [%] |
| 3.9 | 77.0 |
| 7.9 | 25.9 |
| 8.8 | 100.0 |
| 10.4 | 17.5 |
| 10.9 | 20.8 |
| 12.6 | 13.8 |
| 13.5 | 28.6 |
| 13.9 | 12.6 |
| 15.8 | 27.0 |
| 16.4 | 11.1 |
| 17.8 | 15.2 |
| 19.6 | 11.6 |
| 20.3 | 11.2 |
| 21.1 | 25.6 |
| 23.4 | 21.7 |

In one embodiment, the present invention provides a process of preparing form XI of Tigecycline, comprising steps of:
a) dissolving Tigecycline in methylene chloride
b) crystallizing Tigecycline form XI by the addition of n-heptane
c) stirring the solution at room temperature or below to effect complete crystallization
d) optionally isolating crystalline form XI of Tigecycline

For preparing form XI of Tigecycline according to the above process, any other form of Tigecycline may be used, e.g. the amorphous form, crystalline form I or II disclosed in WO 2007/127292 or crystalline form I to V disclosed in WO 2006/128150. In addition, also forms of low crystallinity or mixtures of two or more different forms of Tigecycline may be used. The crystallization step b) of the above process may be facilitated by adding seed crystals of form XI of Tigecycline. The process represents a practical purification method for Tigecycline, because most of the impurities of Tigecycline are soluble in methylene chloride and remain in solution.

Accordingly, form XI of Tigecycline is also a particularly suitable form for the isolation of Tigecycline in the last step of the synthesis of Tigecycline. If, for example, 9-chloroacetaminominocycline is reacted with tert.-butylamine in dimethylacetamide, Tigecycline can be obtained after a simple extractive work up in high yield and in high purity without an additional purification step. Analysis (area %) by HPLC shows a purity of the product of greater than 98.1% with a C₄-epimer content of less than 1.4%.

For the preparation of form XI of Tigecycline room temperature or an elevated temperature may be applied but usually it will be in the range of 10° C. to 40° C. However, it is crucial that solvent and temperature are chosen such that the used form of Tigecycline crystallizes out of the solution, remains in the condition of a suspension and does not dissolve again. It is well within the general knowledge of a person skilled in the art to determine temperature accordingly.

In another embodiment the present invention relates to a method of preparing amorphous Tigecycline by spray drying form XI or another crystalline form of Tigecycline. It has been surprisingly found that spray drying yields amorphous Tigecycline in high purity without a significant increase of the C₄-epimer content although Tetracycline epimerization is known to be temperature dependent (P. H. Yuen, T. D. Sokoloski, J. Pharm. Sci. 1977, (66), 1646-1650). As lyophilization is a cost-intensive process of preparing amorphous Tigecycline the present invention provides a cost effective alternative process of preparing amorphous Tigecycline comprising the steps of:
a) dissolving or slurrying Tigecycline in a suitable solvent
b) spray drying the solution or suspension

Suitable solvents may be polar solvents like water, alcohols (e.g. methanol) and ketones (e.g. acetone). Furthermore methylene chloride is suitable too.

The invention is further described by reference to the following examples. These examples are provided for illustration purposes only and are not intended to be limiting the present invention in any way.

### EXAMPLES

Powder diffractogram of form XI was collected on a Unisantis XMD 300 X-ray powder diffractometer with a position sensitive detector in parallel beam optics using the following acquisition conditions: tube anode: Cu, 40 kV, 0.8 mA; 3-43 degrees theta/2-theta; simultaneous detection of regions of 10 degrees per step with detector resolution 1024, counting time 300 seconds per step. A typical precision of the 2-theta values is in the range±0.2 degrees 2-theta. Thus a diffraction peak that appears at 5.0 degrees 2-theta can appear between 4.8 and 5.2 degrees 2-theta on most X-ray diffractometers under standard conditions.

Infrared spectra were collected on a diamond ATR cell with a Bruker Tensor 27 FTIR spectrometer with 4 cm⁻¹ resolution. A typical precision of the wavenumber values is in the range±2 cm⁻¹. Thus an infrared peak that appears at 1716 cm⁻¹ can appear between 1714 and 1718 cm⁻¹ on most infrared spectrometers under standard conditions.

Differential scanning calorimetry (DSC) was performed on a Netzsch DSC 204 instrument. Samples were heated in 25 μl Al-Pans with loose lids from room temperature to 250° C. at a rate of 10° C./min. Nitrogen (purge rate 20 ml/min) was used as purge gas.

Thermogravimetric analysis (TGA) was performed on a Netzsch STA 409 PC/PG instrument. Samples were heated in an Al₂O₃ crucible from room temperature to 300° C. at a rate of 10° C./min. Nitrogen (purge rate 50 ml/min) was used as purge gas.

Spray drying was performed on a Büchi mini spray dryer 190.

### Example 1

### Preparation of Tigecycline Form XI

25 ml N,N-dimethylacetamide and 10.0 g 9-chloroacetaminominocycline×1.5 TBA (t-butylamine) were put into a three-necked-flask at room temperature. 9.05 g t-butylamine and 3.0 g sodium iodide were added to the suspension and the reaction mixture was stirred for 2 hours at 50° C. Then the reaction mixture was transferred into a Schmizo reactor and diluted with 250 ml methylene chloride and 250 ml water. The pH was adjusted to 8.29 (±0.1) by dropwise adding concentrated hydrochloric acid. After stirring the mixture for 5 to 10 minutes the layers were separated. Before the aqueous layer was washed two times with 250 ml methylene chloride, the pH was adjusted to 8.0±0.1 with 0.1 N sodium hydroxide. The united organic layers were washed three times with 250 ml water and again the pH was adjusted to 8.0 by dropwise adding either 0.1 N hydrochloric acid or 0.1 N sodium hydroxide before washing. Then the organic layer was filtered through a fluted filter and evaporated at 40° C. on the rotavapor. The liquid residue was diluted with 100 ml of methylene chloride and again transferred into a Schmizo reactor. Tigecycline was crystallized within a few minutes by the addition of 120 ml n-heptane. The suspension was stirred for 3 hours at room temperature and over night at 5° C. The solid was filtered off, washed with a cold methylene chloride/n-heptane (3:7) mixture and dried under vacuum over night to obtain 8.2 g (92% of yield) of crystalline form XI of Tigecycline. (HPLC area % 98.8 with a C4-epimer content of 0.6%; GC: 13.21% n-heptane)

### Example 2

### Preparation of Amorphous Tigecycline

883 mg crystalline Tigecycline Form XI were dissolved in 53 ml water at room temperature. The solution was subjected to spray drying in the following setting of spray dryer:

Büchi mini spray dryer 190

| Inlet Temperature | 150° C. |
|---|---|
| Outlet Temperature | 72° C. |
| Aspirator | 100% |
| Pump | 10% |

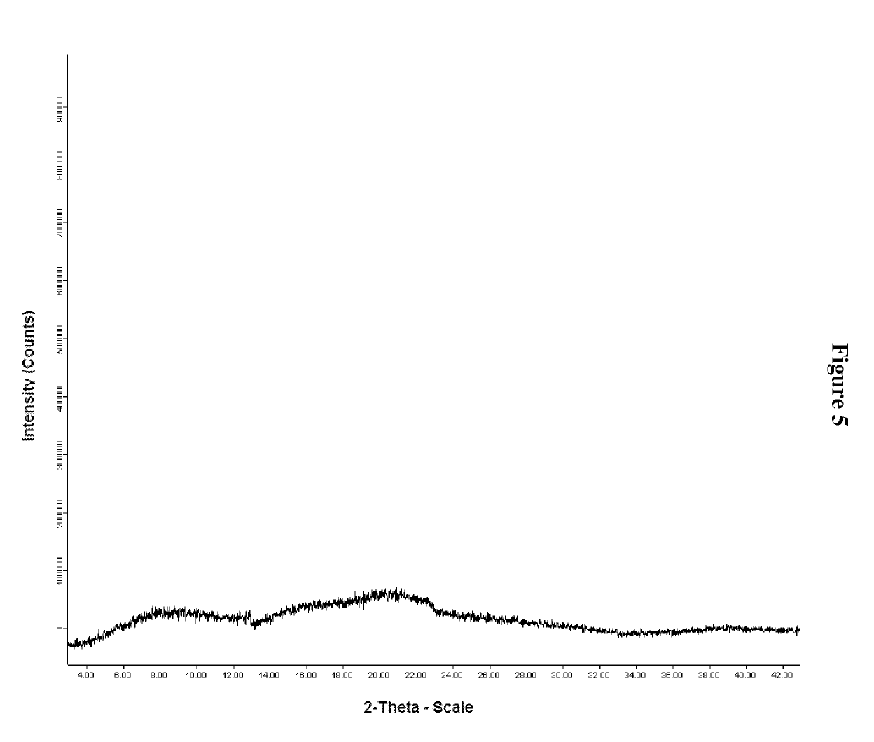

Spray drying resulted in amorphous Tigecycline with an X-ray powder pattern in accordance with FIG. 5.

### Example 3

### Preparation of Amorphous Tigecycline

500 mg crystalline Tigecycline Form XI were dissolved in 75 ml of a methanol/methylene chloride mixture (11:4) at room temperature. The solution is subjected to spray drying in the following setting of spray dryer:

Büchi mini spray dryer 190

| Inlet Temperature | 140° C. |
|---|---|
| Outlet Temperature | 82° C. |
| Aspirator | 100% |
| Pump | 15% |

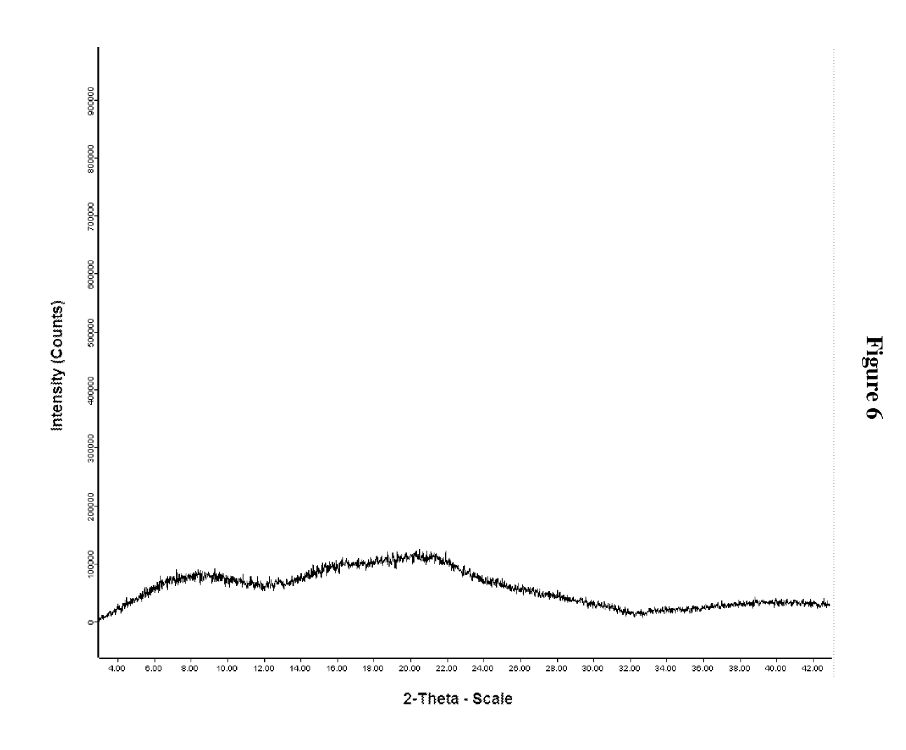

Spray drying resulted in amorphous Tigecycline with an X-ray powder pattern in accordance with FIG. 6.

1-10. (canceled)

11. Crystalline form XI of Tigecycline.

12. The crystalline form XI of Tigecycline according to claim 11, having an X-ray powder diffraction pattern with peaks at 3.9, 7.9, 8.8, 10.4, 10.9, 12.6, 13.5, 13.9, 15.8, 16.4, 17.8, 19.6, 20.3, 21.1 and 23.4±0.2° 2-theta.

13. The crystalline form XI of Tigecycline according to claim 11, having an X-ray powder diffraction pattern substantially in accordance with FIG. 1.

14. The crystalline form XI of Tigecycline according to claim 11, having an Infrared spectrum with peaks at 2957, 2927, 2788, 1675, 1612, 1518, 1281, 1056 and 871±2 cm⁻¹.

15. The crystalline form XI of Tigecycline according to claim 11, having an Infrared spectrum substantially in accordance with FIG. 2.

16. The crystalline form XI of Tigecycline according to claim 11, having an X-ray powder diffraction pattern with a peak at position 8.8°±0.2° 20 having the highest relative intensity.

17. The crystalline form XI of Tigecyline according to claim 11, wherein the crystalline form comprises a n-heptane solvate.

18. A process of preparing form XI of Tigecycline, comprising the steps of:
a) dissolving Tigecycline in methylene chloride to form a solution;
b) adding n-heptane to the solution to crystallize form XI of Tigecycline and form a dispersion;
c) stirring the dispersion at room temperature or below to effect complete crystallization of the form XI of Tigecycline; and
d) optionally isolating the crystalline form XI of Tigecycline.

19. The process according to claim 18, further comprising adding seed crystals of form XI of Tigecycline to step b).

20. The process according to claim 18, wherein the crystalline form XI of Tigecycline is produced without purification steps having a chromatographic purity greater than 98.1% and a C₄ epimer content of less than 1.4%.

21. Method of using form XI of Tigecycline as crystalline pseudo polymorphic form for the isolation of Tigecycline in a last step of a synthesis of Tigecycline.

22. A process of forming amorphous Tigecycline comprising steps of:
a) dissolving form XI of Tigecycline or any other crystalline form of Tigecycline in a suitable solvent; and
b) spray drying the solution to form amorphous Tigecycline.

23. The process according to claim 22, wherein the suitable solvent comprises at least one of water, an alcohol, a ketone, or mixtures thereof.

24. The process according to claim 23, wherein the solvent comprises methanol.

25. The process according to claim 23, wherein the solvent comprises acetone.

26. The process according to claim 23, wherein the solvent comprises methylene chloride.

27. The process according to claim 22, wherein spray drying is performed at 100-150° C.
